(19) United States
(12) Patent Application Publication
(10) Pub. No.: US 2021/0085627 A1
(43) Pub. Date: Mar. 25, 2021

(54) METHODS OF TREATING DISEASES AND DISORDERS USING COMPOSITIONS COMPRISING 18-HEPE, 12-HEPE, OR COMBINATIONS THEREOF

(71) Applicant: Afimmune Limited, Dublin (IE)

(72) Inventors: David Coughlan, Dublin (IE); Moayed Hamza, Dublin (IE)

(21) Appl. No.: 16/582,817

(22) Filed: Sep. 25, 2019

(51) Int. Cl. A61K 31/202; A61K 9/00; A61K 9/48

(52) U.S. Cl. CPC A61K 31/202; A61K 9/48; A61K 9/0014; A61K 9/0053

(57) ABSTRACT

The present disclosure provides compositions comprising 18-HEPE, 12-HEPE, and/or a derivative thereof and methods of using same to treat a variety of diseases and disorders.

### TECHNICAL FIELD

The present application relates generally to compositions comprising 18-HEPE, 12-HEPE or a combination thereof, and to methods of using the same.

### BACKGROUND

18-hydroxy-5Z,8Z,11Z,14Z,16E-eicosapentaenoic acid (“18-HEPE”) and 12-hydroxy-5Z,8Z,10E,14Z,17Z-eicosapentaenoic acid (“12-HEPE”) are metabolites of eicosapentaenoic acid, a polyunsaturated fatty acid known to confer benefits in cardiovascular health.

### SUMMARY

In some aspects, present disclosure relates to compositions comprising 18-HEPE, 12-HEPE, or a combination thereof and to methods of using such composition in the treatment of a variety of diseases and/or disorders.

In another aspect, the present disclosure relates to compositions comprising 18-HEPE, 12-HEPE, and/or derivative thereof.

In some embodiments, the diseases and/or disorders are selected from the group consisting of skin, inflammatory, renal, rheumatic, respiratory, lung, cardiovascular, neurologic, fibrotic, liver, ocular, connective tissue, cancer, peroxisome proliferator activation receptor, urinary, and immunosuppression diseases and/or disorders.

In another embodiment, the skin diseases and/or disorders are selected from the group consisting of acne vulgaris, acne rosacea, atopic dermatitis, psoriasis, pruritus/itch, radiation protection, dry skin, smooth skin, healthy skin, anti-aging, and photoprotection.

In one embodiment, the inflammatory diseases and/or disorders are selected from the group consisting of chronic obstructive pulmonary disease (COPD), asthma, atopic dermatitis, and chronic rhinosinusitis.

In yet another embodiment, the renal diseases and/or disorders are selected from the group consisting of kidney failure, acute kidney injury, chronic kidney disease, end stage renal disease, interstitial nephritis, kidney fibrosis, tubulointerstitial fibrosis, severe interstitial fibrosis, renal dysfunction, renal interstitial fibrosis, and polycystic kidney disease.

In some embodiments, the rheumatic diseases and/or disorders are selected from the group consisting of ankylosing spondylitis, fibromyalgia, gout, infectious arthritis, lupus, osteoarthritis, polymyalgia rheumatica, psoriatic arthritis, reactive arthritis, rheumatoid arthritis, and scleroderma.

In another embodiment, the respiratory and/or lung diseases and/or disorders are selected from the group consisting of inflammatory lung disease, respiratory tract infections, pleural cavity disease, pulmonary vascular disease, pneumonia, pulmonary embolism, lung cancer, idiopathic pulmonary fibrosis, sarcoidosis, mixed connective tissue disease, polymyositis, dermatomyositis, and systemic lupus erythematosus.

In various embodiments, cardiovascular diseases and/or disorders are selected from the group consisting of acute cardiac ischemic events, acute myocardial infarction, angina, arrhythmia, atrial fibrillation, atherosclerosis, arterial fibrillation, cardiac insufficiency, cardiovascular disease, chronic heart failure, chronic stable angina, congestive heart failure, coronary artery disease, coronary heart disease, deep vein thrombosis, diabetes, diabetes mellitus, diabetic neuropathy, diastolic dysfunction in subjects with diabetes mellitus, edema, essential hypertension, eventual pulmonary embolism, fatty liver disease, heart disease, heart failure, homozygous familial hypercholesterolemia (HoFH), homozygous familial sitosterolemia, hypercholesterolemia, hyperlipidemia, hypertension, hypertriglyceridemia, metabolic syndrome, mixed dyslipidemia, moderate to mild heart failure, myocardial infarction, obesity management, paroxysmal atrial/arterial fibrillation/flutter, paroxysmal supraventricular tachycardias (PSVT), particularly severe or rapid onset edema, platelet aggregation, primary hypercholesterolemia, primary hyperlipidemia, pulmonary arterial hypertension, pulmonary hypertension, recurrent hemodynamically unstable ventricular tachycardia (VT), recurrent ventricular arrhythmias, recurrent ventricular fibrillation (VF), ruptured aneurysm, sitosterolemia, stroke, supraventricular tachycardia, symptomatic atrial fibrillation/flutter, tachycardia, type-II diabetes, vascular disease, venous thromboembolism, ventricular arrhythmias, and other cardiovascular events.

In some embodiments, the neurologic diseases and/or disorders are selected from the group consisting of catalepsy, epilepsy, encephalitis, meningitis, migraine, Huntington's, Alzheimer's, Parkinson's, and multiple sclerosis.

In another embodiment, the fibrotic diseases and/or disorders are selected from the group consisting of systemic fibrosis (i.e., radiation fibrosis), liver fibrosis and/or cirrhosis, renal fibrosis, lung fibrosis and/or interstitial lung disease, skin fibrosis, cardiac fibrosis, ocular fibrosis, ocular disease, connective tissue disorders, myelofibrosis, cancers, idiopathic pulmonary fibrosis, and peyronie's disease.

In one embodiment, the liver diseases and/disorders are selected from the group consisting of fatty liver disease, non-alcoholic fatty liver disease (NAFLD), non-alcoholic steatohepatitis (NASH), alcoholic hepatitis, primary sclerosing cholangitis, primary biliary cholangitis, viral hepatitis (Chronic Hepatitis C, HBV), autoimmune hepatitis, Iatrogenic and/or drug induced liver injury, and hepatic veno-occlusive disease.

In some embodiments, the ocular diseases and/or disorders are selected from the group consisting of corneal opacification, glaucoma, age-related macular degeneration (AMD), cataract, and diabetic retinopathy (DR).

In yet another embodiment, the cancer is selected from the group consisting of renal cell carcinoma, hepatocellular carcinoma, cholangiocarcinoma, breast cancer, and cutaneous squamous cell carcinoma.

In one embodiment, the connective tissue diseases and/or disorders are selected from the group consisting of genetic disorders, autoimmune disorders, and cancer.

In another embodiment, the composition is administered to the subject in an amount sufficient to provide up to about 10 g of 18-HEPE, 12-HEPE, and/or a derivative thereof per day.

In some embodiments, the composition is administered in 1 to 8 capsules per day. In one embodiment, each capsule comprises up to about 1 g of 18-HEPE, 12-HEPE, and/or a derivative thereof.

In another embodiment, the derivative thereof is selected from the group consisting of an ester, a conjugate, a salt, or combination thereof.

In one embodiment, the composition is administered to the subject for a period of at least about 1 week, at least about 2 weeks, at least about 4 weeks, at least about 6 weeks, at least about 8 weeks, or at least about 10 weeks.

In one embodiment, the composition is not encapsulated. In yet another embodiment, the composition is encapsulated in a capsule shell. In another embodiment, the composition is in an oral dosage form.

In one embodiment, the composition is administered by oral administration or by topical administration.

These and other embodiments of the invention are described in further detail below.

### DETAILED DESCRIPTION

While the present invention is capable of being embodied in various forms, the description below of several embodiments is made with the understanding that the present disclosure is to be considered as an exemplification of the invention, and is not intended to limit the invention to the specific embodiments illustrated. Headings are provided for convenience only and are not to be construed to limit the invention in any manner. Embodiments illustrated under any heading may be combined with embodiments illustrated under any other heading.

The use of numerical values in the various quantitative values specified in this application, unless expressly indicated otherwise, are stated as approximations as though the minimum and maximum values within the stated ranges were both preceded by the word “about.” In this manner, slight variations from a stated value can be used to achieve substantially the same results as the stated value. Also, the disclosure of ranges is intended as a continuous range including every value between the minimum and maximum values recited, as well as any ranges that can be formed by such values. Also disclosed herein are any and all ratios (and ranges of any such ratios) that can be formed by dividing a recited numeric value into any other recited numeric value. Accordingly, the skilled person will appreciate that many such ratios, ranges, and ranges of ratios can be unambiguously derived from the numerical values presented herein; and, in all instances, such ratios, ranges, and ranges of ratios represent various embodiments of the present invention.

### Definitions

As used herein, “18-HEPE” is 18-hydroxy-5Z,8Z,11Z,14Z,16E-eicosapentaenoic acid and “12-HEPE” is 12-hydroxy-5Z,8Z,10E,14Z,17Z-eicosapentaenoic acid. 18-HEPE and 12-HEPE can be synthesized from eicosapentaenoic acid (“EPA,” eicosa-5,8,11,14,17-pentaenoic acid or 20:5n-3), an omega-3 fatty acid according to methods known in the art. “18-HEPE” or “12-HEPE” refer to 18-HEPE or 12-HEPE in their free acid form (e.g., 18-hydroxy-5Z,8Z,11Z,14Z,16E-eicosapentaenoic acid or 12-hydroxy-5Z,8Z,10E,14Z,17Z-eicosapentaenoic acid) and/or a pharmaceutically acceptable ester, conjugate or salt thereof, derivative, or mixtures of any of the foregoing. The derivative of 18-HEPE or 12-HEPE does not include any derivative compound missing the hydroxy group of 18-HEPE or 12-HEPE.

The term “treatment” in relation a given disease or disorder, includes, but is not limited to, inhibiting the disease or disorder, for example, arresting the development of the disease or disorder; relieving the disease or disorder, for example, causing regression of the disease or disorder; or relieving a condition caused by or resulting from the disease or disorder, for example, relieving or treating symptoms of the disease or disorder.

The term “prevention” in relation to a given disease or disorder means: preventing the onset of disease development if none had occurred, preventing the disease or disorder from occurring in a subject that may be predisposed to the disorder or disease but has not yet been diagnosed as having the disorder or disease, and/or preventing further disease/disorder development if already present.

The terms “pharmaceutical composition” and “composition” can be used interchangeable throughout the present disclosure.

### Compositions

In various embodiments, the present disclosure provides orally and/or topically deliverable compositions comprising 18-HEPE, 12-HEPE, or a combination thereof. The compositions of the invention may also comprise an 18-HEPE and/or 12-HEPE derivative in addition to or instead of 18-HEPE and/or 12-HEPE. Such derivatives include alkyl esters; lower alky esters, such as 18-HEPE and/or 12-HEPE methyl or ethyl ester; or 18-HEPE and/or 12-HEPE in triglyceride form. In one embodiment, the present disclosure provides a composition comprising 18-HEPE, 12-HEPE, and/or derivative thereof encapsulated in a capsule shell.

In one embodiment, the capsule shell comprises gelatin (for example, Gelatin RXL or lime bone gelatin with a lower molecular weight). In another embodiment, the capsule shell comprises Gelatin RXL that has been treated by proteolytic enzyme to cut the gelatin pattern and effectively decrease its molecular weight. In one embodiment, the capsule shell comprises (a) gelatin and (b) plasticizers selected from one or more of d-sorbitol and 1,4-sorbitans. In one embodiment, the gelatin is as described in U.S. Pat. No. 7,485,323, and is hereby incorporated by reference herein in its entirety.

In one embodiment, the plasticizer comprises 1,4-sorbitans in an amount from about 20% to about 30%, for example, about 24% and about 28% (on a dry basis), and a D-sorbitol content of about 30% to about 50%, for example, about 35% to about 45% (on a dry basis).

In some embodiments, the capsule shell further comprises glycerol, purified water, titanium dioxide, medium chain triglycerides and lecithin.

In various embodiments, the 18-HEPE, 12-HEPE, and/or derivative thereof is present in a composition of the invention in an amount of about 50 mg to about 5000 mg, about 75 mg to about 2500 mg, or about 100 mg to about 1000 mg, for example, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1025 mg, about 1050 mg, about 1075 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2025 mg, about 2050 mg, about 2075 mg, about 2100 mg, about 2125 mg, about 2150 mg, about 2175 mg, about 2200 mg, about 2225 mg, about 2250 mg, about 2275 mg, about 2300 mg, about 2325 mg, about 2350 mg, about 2375 mg, about 2400 mg, about 2425 mg, about 2450 mg, about 2475 mg, or about 2500 mg. In any such embodiment, the composition can further comprise 18-HEPE esters of D-Sorbitol and 1,4-sorbitan, 12-HEPE esters of D-Sorbitol and 1,4-sorbitan, and/or derivative thereof.

A composition of the invention comprises a therapeutically effective amount of a salt of 18-HEPE, 12-HEPE, and/or derivative thereof. The salt form of 18-HEPE, 12-HEPE, and/or derivative thereof may be the sole significant active ingredient in that composition and in the methods and uses as stated herein. Alternatively, the salt form of 18-HEPE, 12-HEPE, and/or derivative thereof may be combined for co-formulation or co-administration with other agents for treating a disease and/or disorder. If an additional active agent is to be used, the salt form of 18-HEPE, 12-HEPE, and/or derivative thereof can be co-formulated as a single dosage unit or can be formulated as two to a plurality of dosage units for coordinated, combination or concomitant administration. In some embodiments, the 18-HEPE, 12-HEPE, and/or derivative thereof are in the lysine salt form.

In one embodiment, the composition comprises at least about 50%, at least about 60%, at least about 70%, at least about 80% or at least about 90%, by weight of the salt form of 18-HEPE, 12-HEPE, and/or derivative thereof.

In another embodiment, the salt form of 18-HEPE, 12-HEPE, and/or derivative thereof is present in a composition of the invention in an amount of about 1 mg to about 10,000 mg, about 25 mg to about 7500 mg, about 25 mg to about 5000 mg, about 50 mg to about 5000 mg, about 50 mg to about 3000 mg, about 75 mg to about 2500 mg, or about 100 mg to about 1000 mg, for example about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1025 mg, about 1050 mg, about 1075 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2025 mg, about 2050 mg, about 2075 mg, about 2100 mg, about 2125 mg, about 2150 mg, about 2175 mg, about 2200 mg, about 2225 mg, about 2250 mg, about 2275 mg, about 2300 mg, about 2325 mg, about 2350 mg, about 2375 mg, about 2400 mg, about 2425 mg, about 2450 mg, about 2475 mg, about 2500 mg, 2525 mg, about 2550 mg, about 2575 mg, about 2600 mg, about 2625 mg, about 2650 mg, about 2675 mg, about 2700 mg, about 2725 mg, about 2750 mg, about 2775 mg, about 2800 mg, about 2825 mg, about 2850 mg, about 2875 mg, about 2900 mg, about 2925 mg, about 2950 mg, about 2975 mg, about 3000 mg, about 3025 mg, about 3050 mg, about 3075 mg, about 3100 mg, about 3125 mg, about 3150 mg, about 3175 mg, about 3200 mg, about 3225 mg, about 3250 mg, about 3275 mg, about 3300 mg, about 3325 mg, about 3350 mg, about 3375 mg, about 3400 mg, about 3425 mg, about 3450 mg, about 3475 mg, about 3500 mg, about 3525 mg, about 3550 mg, about 3575 mg, about 3600 mg, about 3625 mg, about 3650 mg, about 3675 mg, about 3700 mg, about 3725 mg, about 3750 mg, about 3775 mg, about 3800 mg, about 3825 mg, about 3850 mg, about 3875 mg, about 3900 mg, about 3925 mg, about 3950 mg, about 3975 mg, about 4000 mg, about 4025 mg, about 4050 mg, about 4075 mg, about 4100 mg, about 4125 mg, about 4150 mg, about 4175 mg, about 4200 mg, about 4225 mg, about 4250 mg, about 4275 mg, about 4300 mg, about 4325 mg, about 4350 mg, about 4375 mg, about 4400 mg, about 4425 mg, about 4450 mg, about 4475 mg, about 4500 mg, about 4525 mg, about 4550 mg, about 4575 mg, about 4600 mg, about 4625 mg, about 4650 mg, about 4675 mg, about 4700 mg, about 4725 mg, about 4750 mg, about 4775 mg, about 4800 mg, about 4825 mg, about 4850 mg, about 4875 mg, about 4900 mg, about 4925 mg, about 4950 mg, about 4975 mg, about 5000 mg, about 5025 mg, about 5050 mg, about 5075 mg, about 5100 mg, about 5125 mg, about 5150 mg, about 5175 mg, about 5200 mg, about 5225 mg, about 5250 mg, about 5275 mg, about 5300 mg, about 5325 mg, about 5350 mg, about 5375 mg, about 5400 mg, about 5425 mg, about 5450 mg, about 5475 mg, about 5500 mg, about 5525 mg, about 5550 mg, about 5575 mg, about 5600 mg, about 5625 mg, about 5650 mg, about 5675 mg, about 5700 mg, about 5725 mg, about 5750 mg, about 5775 mg, about 5800 mg, about 5825 mg, about 5850 mg, about 5875 mg, about 5900 mg, about 5925 mg, about 5950 mg, about 5975 mg, about 6000 mg, about 6025 mg, about 6050 mg, about 6075 mg, about 6100 mg, about 6125 mg, about 6150 mg, about 6175 mg, about 6200 mg, about 6225 mg, about 6250 mg, about 6275 mg, about 6300 mg, about 6325 mg, about 6350 mg, about 6375 mg, about 6400 mg, about 6425 mg, about 6450 mg, about 6475 mg, about 6500 mg, about 6525 mg, about 6550 mg, about 6575 mg, about 6600 mg, about 6625 mg, about 6650 mg, about 6675 mg, about 6700 mg, about 6725 mg, about 6750 mg, about 6775 mg, about 6800 mg, about 6825 mg, about 6850 mg, about 6875 mg, about 6900 mg, about 6925 mg, about 6950 mg, about 6975 mg, about 7000 mg, about 7025 mg, about 7050 mg, about 7075 mg, about 7100 mg, about 7125 mg, about 7150 mg, about 7175 mg, about 7200 mg, about 7225 mg, about 7250 mg, about 7275 mg, about 7300 mg, about 7325 mg, about 7350 mg, about 7375 mg, about 7400 mg, about 7425 mg, about 7450 mg, about 7475 mg, about 7500 mg, about 7525 mg, about 7550 mg, about 7575 mg, about 7600 mg, about 7625 mg, about 7650 mg, about 7675 mg, about 7700 mg, about 7725 mg, about 7750 mg, about 7775 mg, about 7800 mg, about 7825 mg, about 7850 mg, about 7875 mg, about 7900 mg, about 7925 mg, about 7950 mg, about 7975 mg, about 8000 mg, about 8025 mg, about 8050 mg, about 8075 mg, about 8100 mg, about 8125 mg, about 8150 mg, about 8175 mg, about 8200 mg, about 8225 mg, about 8250 mg, about 8275 mg, about 8300 mg, about 8325 mg, about 8350 mg, about 8375 mg, about 8400 mg, about 8425 mg, about 8450 mg, about 8475 mg, about 8500 mg, about 8525 mg, about 8550 mg, about 8575 mg, about 8600 mg, about 8625 mg, about 8650 mg, about 8675 mg, about 8700 mg, about 8725 mg, about 8750 mg, about 8775 mg, about 8800 mg, about 8825 mg, about 8850 mg, about 8875 mg, about 8900 mg, about 8925 mg, about 8950 mg, about 8975 mg, about 9000 mg, about 9025 mg, about 9050 mg, about 9075 mg, about 9100 mg, about 9125 mg, about 9150 mg, about 9175 mg, about 9200 mg, about 9225 mg, about 9250 mg, about 9275 mg, about 9300 mg, about 9325 mg, about 9350 mg, about 9375 mg, about 9400 mg, about 9425 mg, about 9450 mg, about 9475 mg, about 9500 mg, about 9525 mg, about 9550 mg, about 9575 mg, about 9600 mg, about 9625 mg, about 9650 mg, about 9675 mg, about 9700 mg, about 9725 mg, about 9750 mg, about 9775 mg, about 9800 mg, about 9825 mg, about 9850 mg, about 9875 mg, about 9900 mg, about 9925 mg, about 9950 mg, about 9975 mg, or about 10,000 mg.

In one embodiment, the salt form of 18-HEPE, 12-HEPE, and/or derivative thereof present in a composition of the invention comprises at least 90% by weight of the salt form of 18-HEPE, 12-HEPE, and/or derivative thereof. Compositions containing the salt form of 18-HEPE, 12-HEPE, and/or derivative thereof can comprise even higher purity, for example at least about 91% by weight, at least about 92% by weight, at least about 93% by weight, at least about 94% by weight, at least about 95% by weight, at least about 96% by weight or at least about 97% by weight of the salt form of 18-HEPE, 12-HEPE, and/or derivative thereof.

In one embodiment, a composition of the invention contains not more than about 10%, not more than about 9%, not more than about 8%, not more than about 7%, not more than about 6%, not more than about 5%, not more than about 4%, not more than about 3%, not more than about 2%, not more than about 1%, or not more than about 0.5%, by weight of total fatty acids, of fatty acids other than 18-HEPE, 12-HEPE, and/or derivative thereof.

In another embodiment, 18-HEPE, 12-HEPE, and/or derivative thereof represents at least about 30%, about 40%, about 50%, at least about 60%, at least about 70%, at least about 80%, at least about 90%, at least about 95%, at least about 97%, at least about 98%, at least about 99%, or 100%, by weight, of all fatty acids present in a composition of the invention.

In one embodiment, a composition of the invention when placed in a standard disintegration test, for example, as set forth in USP 2040 (Disintegration and Dissolution of Dietary Supplements) with water as the Medium has a 18-HEPE, 12-HEPE, and/or derivative thereof release rate less than about 60 minutes, less than about 50 minutes, less than about 40 minutes, less than about 30 minutes, or less than 20 minutes after storage for about 1 month, about 2 months, or about 3 months at 40° C./75% RH.

In one embodiment, after storage for about 1 month, about 2 months, about 3 months, or about 6 months at 40° C./75% RH, a composition of the invention comprises less than about 5% 18-HEPE esters and/or 12-HEPE esters by weight of all fatty acids, less than about 4% 18-HEPE esters and/or 12-HEPE esters by weight of all fatty acids, less than about 3% 18-HEPE esters and/or 12-HEPE esters by weight of all fatty acids, less than about 2% 18-HEPE esters and/or 12-HEPE esters by weight of all fatty acids, or less than about 1% 18-HEPE esters and/or 12-HEPE esters by weight of all fatty acids.

### Additional Active Agents

In one embodiment, the composition further comprises one or more additional active agent(s). In one embodiment, the composition comprises an amount of the additional active agent that is less than the generally recognized therapeutically effective amount for that agent. In one embodiment, the composition comprises an amount of the additional active agent that is equal to or greater than the generally recognized therapeutically effective amount for that agent. If an additional active agent is to be used, the 18-HEPE, 12-HEPE, and/or derivative thereof can be co-formulated as a single dosage unit or can be formulated as two to a plurality of dosage units for coordinated, combination or concomitant administration.

EPA itself has beneficial properties in treating various diseases and disorders and it is possible to combine the 18-HEPE, 12-HEPE, and/or derivative thereof with EPA in an alternative embodiment.

In one embodiment, 18-HEPE, 12-HEPE, and/or derivative thereof and one or more active agent(s) are present in a composition of the disclosure, or are co-administered in a weight ratio of 18-HEPE, 12-HEPE, and/or derivative thereof: additional agent of about 1:1000 to about 1000:1, about 1:500 to about 500:1, about 1:100 to about 100:1, about 1:50 to about 50:1, about 1:25 to about 25:1, about 1:10 to about 10:1, about 1:5 to about 5:1, about 1:4 to about 4:1 about 1:3 to about 3:1, about 1:2 to about 2:1 or about 1:1.

A composition for use in accordance with the disclosure can be formulated as one or more dosage units. The terms “dose unit” and “dosage unit” herein refer to a portion of a composition that contains an amount of a therapeutic agent suitable for a single administration to provide a therapeutic effect. Such dosage units may be administered one to a plurality (e.g., 1 to about 10, 1 to 8, 1 to 6, 1 to 4 or 1 to 2) of times per day, or as many times as needed to elicit a therapeutic response.

In some embodiments, compositions of the disclosure are in the form of orally deliverable dosage forms or units. Non-limiting examples of suitable dosage forms include tablets (e.g., suspension tablets, bite suspension tablets, rapid dispersion tablets, chewable tablets, etc), caplets, capsules (e.g., a soft or a hard gelatin capsule or HPMC capsule), lozenges, sachets, cachets, troches, pellets, suspension, elixirs, syrups or any other solid dosage form reasonably adapted for oral administration. The terms “oral delivery” and “oral administration” herein include any form of delivery wherein the agent or composition is placed in the mouth of the subject under treatment, whether swallowed or not. This therefore includes buccal and sublingual administration, as well as esophageal administration.

Alternatively, compositions of the disclosure can also be formulated for rectal, topical, or parenteral (e.g., subcutaneous, intramuscular, intravenous and intradermal or infusion) delivery.

In some embodiments, the composition is in a form suitable for topical administration. In various embodiments, the invention provides pharmaceutical compositions, for example topically deliverable pharmaceutical compositions, comprising 18-HEPE, 12-HEPE, and/or derivative thereof. In yet another embodiment, present disclosure provides topical compositions comprising, for example, an amount (e.g., a therapeutically effective amount) 18-HEPE, 12-HEPE, and/or derivative thereof.

In discussing the amount of 18-HEPE, 12-HEPE, and/or derivative thereof in a composition of the disclosure, this may be split over several dosage forms. There is a limit as to the size for oral administration. If a subject is to be administered about 1 to about 4 g 18-HEPE, 12-HEPE, and/or derivative thereof a day, this may be by up to 4 capsules, each providing about 1 g of 18-HEPE, 12-HEPE, and/or derivative thereof.

Compositions of the disclosure can be in the form of liquid dosage forms or dose units to be imbibed directly or they can be mixed with food or beverage prior to ingestion. Non-limiting examples of suitable liquid dosage forms include solutions, suspensions, elixirs, syrups, liquid aerosol formulations, and the like.

In another embodiment, compositions of the disclosure comprise one or more pharmaceutically acceptable excipients. The term “pharmaceutically acceptable excipient” herein means any substance, not itself a therapeutic agent, used as a carrier or vehicle for delivery of a therapeutic agent to a subject or added to a composition to improve its handling or storage properties or to permit or facilitate formation of a unit dose of the composition, and that does not produce unacceptable toxicity or interaction with other components in the composition. By way of example only, a pharmaceutical composition according to the present disclosure may comprise one or more of: antioxidants, surfactants, preservatives, flavoring agents, co-solvents, viscosity aids, suspension aids, and lipophilic phases.

In one embodiment, the pharmaceutical composition comprises one or more antioxidants such as ascorbic acid, palmitic acid, ascorbyl palmitate, a-tocopherol, idebenone, ubiquinone, ferulic acid, coenzyme Q10, lycopene, green tea, catechins, epigallocatechin 3-gallate (EGCG), green tea polyphenols (GTP), silymarin, coffeeberry, resveratrol, grape seed, pomegranate extracts, genisten, pycnogenol, niacinamide, and the like. In one embodiment, the pharmaceutical composition comprises about 0.01 wt. % to about 2 wt. % of an antioxidant, for example about 0.01 wt. %, about 0.02 wt. %, about 0.03 wt. %, about 0.04 wt. %, about 0.05 wt. %, about 0.06 wt. %, about 0.07 wt. %, about 0.08 wt. %, about 0.09 wt. %, about 0.1 wt. %, about 0.11 wt. %, about 0.12 wt. %, about 0.13 wt. %, about 0.14 wt. %, about 0.15 wt. %, about 0.16 wt. %, about 0.17 wt. %, about 0.18 wt. %, about 0.19 wt. %, about 0.2 wt. %, about 0.21 wt. %, about 0.22 wt. %, about 0.23 wt. %, about 0.24 wt. %, about 0.25 wt. %, about 0.26 wt. %, about 0.27 wt. %, about 0.28 wt. %, about 0.29 wt. %, about 0.3 wt. %, about 0.31 wt. %, about 0.32 wt. %, about 0.33 wt. %, about 0.34 wt. %, about 0.35 wt. %, about 0.36 wt. %, about 0.37 wt. %, about 0.38 wt. %, about 0.39 wt. %, about 0.4 wt. %, about 0.41 wt. %, about 0.42 wt. %, about 0.43 wt. %, about 0.44 wt. %, about 0.45 wt. %, about 0.46 wt. %, about 0.47 wt. %, about 0.48 wt. %, about 0.49 wt. %, about 0.5 wt. %, about 0.51 wt. %, about 0.52 wt. %, about 0.53 wt. %, about 0.54 wt. %, about 0.55 wt. %, about 0.56 wt. %, about 0.57 wt. %, about 0.58 wt. %, about 0.59 wt. %, about 0.6 wt. %, about 0.61 wt. %, about 0.62 wt. %, about 0.63 wt. %, about 0.64 wt. %, about 0.65 wt. %, about 0.66 wt. %, about 0.67 wt. %, about 0.68 wt. %, about 0.69 wt. %, about 0.7 wt. %, about 0.71 wt. %, about 0.72 wt. %, about 0.73 wt. %, about 0.74 wt. %, about 0.75 wt. %, about 0.76 wt. %, about 0.77 wt. %, about 0.78 wt. %, about 0.79 wt. %, about 0.8 wt. %, about 0.81 wt. %, about 0.82 wt. %, about 0.83 wt. %, about 0.84 wt. %, about 0.85 wt. %, about 0.86 wt. %, about 0.87 wt. %, about 0.88 wt. %, about 0.89 wt. %, about 0.9 wt. %, about 0.91 wt. %, about 0.92 wt. %, about 0.93 wt. %, about 0.94 wt. %, about 0.95 wt. %, about 0.96 wt. %, about 0.97 wt. %, about 0.98 wt. %, about 0.99 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, or about 2 wt. % of the one or more antioxidant.

### Methods

### Skin Disorders and Diseases

Any composition of the invention, including compositions described herein above or compositions that can for formulated from combining various embodiments of the present disclosure, can be used in treatment or prevention of skin disorders and/or diseases including acne vulgaris, acne rosacea, atopic dermatitis, psoriasis, pruritus/itch, radiation protection, dry skin, smooth skin, healthy skin, anti-aging, and photoprotection.

In some embodiments, the compositions can be used in treatment or prevention of skin inflammation including rashes, hives, blisters and/or wheals and may be caused by eczema, exposure to radiation, autoimmune diseases, and/or uremic pruritis. In another embodiment, the skin inflammation is caused by atopic eczema, contact dermatitis, psoriasis or uremic pruritis. In yet another embodiment, the skin inflammation is caused by exposure of the skin to electromagnetic radiation. This includes, for example, exposure to sunlight, heat, X-rays or radioactive materials. Thus, in this embodiment, the compound of the present invention is used to treat sunburn.

In some embodiments, the compositions can be used in treatment or prevention eczema. Eczema includes atopic eczema (atopic dermatitis), contact dermatitis, xerotic eczema, seborrhoeic dermatitis, dyshydrosis, discoid eczema, venous eczema, dermatitis herpetiformus, neurodermatitis, and autoeczematisation. Atopic eczema is primarily aggravated by contact with or intake of allergens, which include animal hair and dander, food allergens, for example nuts or shellfish, or drugs, for example penicillin. Contact dermatitis includes allergic contact dermatitis, irritant contact dermatitis and photocontact dermatitis.

In some embodiments, the compositions can be used in treatment or prevention of atopic dermatitis, wherein administration of the composition to a subject in need thereof, reduces at least one of an investigator global assessment (IGA) level, eczema area and severity index (EASI) score, a percentage of area of an anatomical site affected by atopic dermatitis, Scoring atopic dermatitis (SCORAD), a body surface area affected by atopic dermatitis, or visual analog scale (VAS) pruritus score. In one embodiment, the subject is determined to have a low baseline eosinophil count as compared to a reference level. In one embodiment, the subject is determined to have a low baseline eosinophil count prior to administration of the composition. In yet another embodiment, the subject is a pediatric subject. In some embodiments, the subject is an adult subject.

The term “reference level” includes, but is not limited to, a level from a sample collected from a healthy patient. A reference level can also be determined from a plurality of samples collected from a population of healthy patients. As one example, a low eosinophil cell count can be determined based on an eosinophil cell count determined from a population of healthy patients, or a subset of healthy patients, for example, healthy patients of a particular ethnicity. In other embodiments, the reference level is a value determined from a sample collected at an earlier time point (e.g., 1 day, 3 days, 1 week, 1 month, 3 months, 6 months, 12 months, or more) from the same patient that is undergoing treatment. In some embodiments, the reference level may be based on values known by those of skill in the art or developed by a medical agency.

### Inflammatory Diseases and Disorders

Any composition of the invention, including compositions described herein above or compositions that can for formulated from combining various embodiments of the present disclosure, can be used in treatment or prevention of inflammatory diseases and/or disorders including chronic obstructive pulmonary disease (COPD), asthma, atopic dermatitis, chronic rhinosinusitis, or a combination thereof.

In some embodiments, the compositions can be used in treatment or prevention of COPD. COPD is a chronic inflammatory lung disease the obstructs airflow from the lungs. In some embodiments, administration of the composition to a subject having COPD relieves breathing difficulties, coughing, mucus production, and wheezing. In some embodiments, the subject has a normal eosinophil count.

In yet another embodiment, the compositions can be used in treatment or prevention of topic dermatitis in a subject having moderate to severe atopic dermatitis. In some embodiments, the subject has a normal eosinophil count.

In one embodiment, the compositions can be used in treatment or prevention of chronic rhinosinusitis including chronic sinusitis with nasal polyps (CRSwNP). Chronic rhinosinusitis is an inflammatory disease of the nose and sinus mucosa. In some embodiments, administration of the composition to a subject having chronic rhinosinusitis relieves headache pain, facial fanny, runny nose, and nasal congestion. In some embodiments, the subject has a normal eosinophil count. In some embodiments, the CRSwNP include a non-eosinophilic subtype.

In some embodiments, the compositions can be used in treatment or prevention of asthma. Asthma causes airways to narrow and swell and results in the production of extra mucus. In some embodiments, administration of the composition to a subject having asthma relieves coughing, wheezing, and shortness of breath. In some embodiments, the asthma is adult-onset asthma, or late-onset asthma. In another embodiment, the asthma is childhood-onset asthma.

In some embodiments, the compositions can be used in treatment or prevention of one or more atopic conditions. In one embodiment, the one or more atopic conditions include atopic dermatitis. In yet another embodiment, the one or more atopic conditions includes vasculitides including large vessel vasculitis (LW), medium vessel vasculitis (MW), small vessel vasculitis (SW), variable vessel vasculitis (WV), vingle-organ vasculitis (SOV) and vasculitis associated with systemic disease.

In some embodiments, the normal eosinophil count is 0.3×10⁹/L. In one embodiment, the subject has eosinophil levels that are greater than normal eosinophil levels, less than normal eosinophil levels, or a combination thereof.

In some embodiments, administration of the composition to the subject having COPD, asthma, atopic dermatitis, chronic rhinosinusitis, or a combination thereof reduces at least one of an IGA level, EASI, a percentage of area of an anatomical SCORAD, a body surface area affected by atopic dermatitis, a dermatology life quality Index (DLQI), a patient-oriented eczema measure (POEM), or VAS pruritus score.

### Urinary Disorders and Diseases

Any composition of the invention, including compositions described herein above or compositions that can for formulated from combining various embodiments of the present disclosure, can be used in treatment or prevention of urinary diseases and/or disorders including bladder cancer, cystocele, hematuria, interstitial cystitis, neurogenic bladder, Peyronie's disease, prostate disease, incontinence, urinary tract infection, and vasicoureteral reflux.

### Renal Diseases and Disorders

Any composition of the invention, including compositions described herein above or compositions that can for formulated from combining various embodiments of the present disclosure, can be used in treatment or prevention of renal diseases and/or disorders including kidney failure, acute kidney injury, chronic kidney disease, end stage renal disease, interstitial nephritis, kidney fibrosis, tubulointerstitial fibrosis, severe interstitial fibrosis, renal dysfunction, renal interstitial fibrosis, and polycystic kidney disease.

In some embodiments, the compositions can be used in the treatment or prevention of glomerular diseases and/or disorders. Non-limiting examples of glomerular diseases and/or disorders include focal segmental glomerulosclerosis (FSGS), IgA nephropathy, crescentic glomerulonephritis, lupus nephritis, tubulointerstitial disease, osteoarthritis, polymyalgia rheumatica, psoriatic arthritis, and diabetic nephropathy. In some embodiments, the compositions can be used in the treatment or prevention of iatrogenic nephropathy and/or renal ischemia.

In another embodiment, the compositions can be used in treatment or prevention of renal diseases and/or disorders in a subject having at least one risk factor for a renal disease and/or disorder including diabetes, high blood pressure, cardiovascular disease, glomerulonephritis, and polycystic kidney disease.

### Rheumatic Diseases and Disorders

Any composition of the invention, including compositions described herein above or compositions that can for formulated from combining various embodiments of the present disclosure, can be used in treatment or prevention of rheumatic diseases and/or disorders including ankylosing spondylitis, fibromyalgia, gout, infectious arthritis, lupus, osteoarthritis, polymyalgia rheumatica, psoriatic arthritis, reactive arthritis, rheumatoid arthritis, and scleroderma.

### Respiratory and Lung Diseases and Disorders

Any composition of the invention, including compositions described herein above or compositions that can for formulated from combining various embodiments of the present disclosure, can be used in treatment or prevention of respiratory diseases and/or disorders including inflammatory lung disease, lung disease or disorder, respiratory tract infections, pleural cavity disease, pulmonary vascular disease, pneumonia, pulmonary embolism, and lung cancer.

In some embodiments, the respiratory diseases and/or disorders are selected from the group consisting of COPD or asthma. In some embodiments, the disease or disorder is COPD. In some embodiments, the disease or disorder is asthma.

In another embodiment, the composition can be used for treating respiratory diseases and/or disorders by reducing the symptoms of, slowing progression of or promoting regression of a lung disease or disorder such as asthma or COPD in a subject. In some embodiments, the composition can be used to reduce bronchoconstriction in a subject.

In some embodiments, the composition can be used in the treatment or prevention of lung diseases and/or disorders selected from the group consisting of idiopathic pulmonary fibrosis, sarcoidosis, mixed connective tissue disease, polymyositis, dermatomyositis, and systemic lupus erythematosus.

In some embodiments, the composition is administered to a lung of a subject having a respiratory disease or disorder by a dry-powder inhaler. In some embodiments, the composition is administered to a lung of the subject by metered dose inhaler. In some embodiments, the composition is in powder form. In some embodiments, the composition further comprises a propellant. In another embodiment, the composition is delivered to a lung of the subject by nebulizer. In some embodiments, the composition is a solution comprising a solvent system. In some embodiments, the solvent system comprises an alcohol and/or a polyol. In some embodiments, the solvent system does not include an alcohol or a polyol. In some embodiments, the composition is a suspension.

### Cardiovascular Diseases and Disorders

Any composition of the invention, including compositions described herein above or compositions that can for formulated from combining various embodiments of the present disclosure, can be used in treatment or prevention of cardiovascular diseases and/or disorders including acute cardiac ischemic events, acute myocardial infarction, angina, arrhythmia, atrial fibrillation, atherosclerosis, arterial fibrillation, cardiac insufficiency, cardiovascular disease, chronic heart failure, chronic stable angina, congestive heart failure, coronary artery disease, coronary heart disease, deep vein thrombosis, diabetes, diabetes mellitus, diabetic neuropathy, diastolic dysfunction in subjects with diabetes mellitus, edema, essential hypertension, eventual pulmonary embolism, fatty liver disease, heart disease, heart failure, homozygous familial hypercholesterolem ia (HoFH), homozygous familial sitosterolemia, hypercholesterolemia, hyperlipidemia, hypertension, hypertriglyceridemia, metabolic syndrome, mixed dyslipidemia, moderate to mild heart failure, myocardial infarction, obesity management, paroxysmal atrial/arterial fibrillation/flutter, paroxysmal supraventricular tachycardias (PSVT), particularly severe or rapid onset edema, platelet aggregation, primary hypercholesterolemia, primary hyperlipidemia, pulmonary arterial hypertension, pulmonary hypertension, recurrent hemodynamically unstable ventricular tachycardia (VT), recurrent ventricular arrhythmias, recurrent ventricular fibrillation (VF), ruptured aneurysm, sitosterolemia, stroke, supraventricular tachycardia, symptomatic atrial fibrillation/flutter, tachycardia, type-II diabetes, vascular disease, venous thromboembolism, ventricular arrhythmias, and other cardiovascular events.

Any composition of the invention, including compositions described herein above or compositions that can for formulated from combining various embodiments of the present disclosure, can be used in treatment or prevention of metabolic syndrome or cardiometabolic disease. Non-limiting examples of cardiometabolic diseases include dyslipidemia, hyperlipidemia, hypercholesterolemia, hypertriglyceridemia, primary hypercholesterolemia, primary hyperlipidemia, common primary hyperlipidemia, common hypercholesterolemia, familial hyperlipidemia, familial primary hyperlipidemia, familial hypercholesterolemia, familial hypertriglyceridemia, familial combined hyperlipidemia, familial defective apolipoprotein b-100, secondary hyperlipidemia, mixed hyperlipidemia, cardiovascular disease, residual cardiovascular risk, prevention of atherosclerotic plaque formation/progression, microvascular disease, macrovascular disease, atherosclerosis, coronary atherosclerosis, diastolic dysfunction, reduction of cardiovascular risk, prevention of major coronary events, prevention of major adverse cardiovascular events, prevention of ischemic events, secondary/primary prevention of cardiovascular events, prevention of cardiovascular death, myocardial infarction, stroke, angina, restoration of normal endothelial function, diabetes, diabetes mellitus, insulin resistance, hyperinsulinemia, hyperglycemia, dysglycemia, induction of glycemic control, impaired glucose tolerance, and impaired fasting glucose. In one embodiment, the microvascular disease is retinopathy, nephropathy, neuropathy, or combination thereof. In another embodiment, the macrovascular disease is stroke, peripheral vascular disease, limb ischemia, heart disease, or combination thereof.

In some embodiments, the composition can be used in impairing insulin sensitivity. In some embodiments, the subject is sensitive to insulin and/or is at risk for developing insulin sensitivity.

### Neurologic Diseases and Disorders

Any composition of the invention, including compositions described herein above or compositions that can for formulated from combining various embodiments of the present disclosure, can be used in treatment or prevention of neurologic diseases and/or disorders including catalepsy, epilepsy, encephalitis, meningitis, migraine, Huntington's, Alzheimer's, Parkinson's, and multiple sclerosis.

In some embodiments, the composition can be used in treatment or prevention of central nervous system (CNS) diseases. In some embodiments, is identified as having an increased risk of developing an CNS disease before administration of the composition. In some embodiments, the subject is identified as having a CNS disease by a screening for a genetic mutation in a nucleic acid molecule associated with the subject. In some embodiments, the subject is identified as having a CNS disease through an analysis of blood and/or serum associated with the subject. In some embodiments, the subject is identified as having a CNS disease from a tissue associated with the subject. In some embodiments, the subject is identified as having a CNS disease by an analysis a histological tissue sample (e.g., a biopsy) associated with the subject.

### Fibrotic Diseases and Disorders

Any composition of the invention, including compositions described herein above or compositions that can for formulated from combining various embodiments of the present disclosure, can be used in treatment or prevention of fibrotic diseases and/or disorders including systemic fibrosis (i.e., radiation fibrosis), liver fibrosis and/or cirrhosis, renal fibrosis, lung fibrosis and/or interstitial lung disease, skin fibrosis, cardiac fibrosis, ocular fibrosis, ocular disease, connective tissue disorders, myelofibrosis, cancers, idiopathic pulmonary fibrosis, peyronie's disease, and other related fibrotic diseases. In one embodiment the fibrosis is associated with an organ or tissue associated with a lung, a liver, a heart, a kidney, one or more eyes, mediastinum, bone marrow, retroperitoneum, skin, an intestine, a joint, a reproductive organ, or a combination thereof.

In one embodiment, the systemic fibrosis is radiation fibrosis. In one embodiment, the renal fibrosis is glomerular diseases, tubulointerstitial disease, iatrogenic nephropathy, and/or renal ischemia. In one embodiment, the glomerular diseases include but are not limited to focal segmental glomerulosclerosis, IgA nephropathy, crescentic glomerulonephritis, lupus nephritis, and diabetic nephropathy. In one embodiment, lung fibrosis further comprises idiopathic pulmonary fibrosis, scleroderma, radiation fibrosis, iatrogenic, sarcoidosis, mixed connective tissue disease, polymyositis, dermatomyositis, interstitial lung disease, and/or systemic lupus erythematosus. In one embodiment, skin fibrosis further comprises scleroderma, systemic sclerosis, nephrogenic fibrosing dermopathy, mixed connective tissue disease, scleromyxedema, scleroderma, eosinophilic fasciitis, and/or iatrogenic fibrosis.

### Liver Diseases and Disorders

Any composition of the invention, including compositions described herein above or compositions that can for formulated from combining various embodiments of the present disclosure, can be used in treatment or prevention of liver diseases and/or disorders such as fatty liver disease. In some embodiments, the fatty liver disease is non-alcoholic fatty liver disease (NAFLD) and/or non-alcoholic steatohepatitis (NASH). In some embodiments, the liver disease and/or disorder is cirrhosis.

Non-limiting examples of liver fibrosis and/or cirrhosis related diseases include NAFLD, alcoholic hepatitis, primary sclerosing cholangitis, primary biliary cholangitis, viral hepatitis (Chronic Hepatitis C, HBV), autoimmune hepatitis, Iatrogenic and/or drug induced liver injury, and/or hepatic veno-occlusive disease.

In some embodiments, the compositions can be used in reducing a NAFLD activity score (NAS) in a subject. In some embodiments, a NAS of the subject is determined before and after administration of the composition to the subject. In some embodiments, the NAS a period of time after administration of the composition is reduced.

In some embodiments, the compositions can be used in the treatment or prevention of cholestatic liver disease including primary biliary cholangitis (PBC), primary sclerosing cholangitis (PSC), progressive familial intrahepatic cholestasis, or combination thereof. In yet another embodiment, the cholestatic liver disease is caused by a drug induced liver injury, total parenteral nutrition (TPN), viral and alcoholic hepatitis, cholestasis secondary to systemic diseases, graft dysfunction, post liver transplant cholestasis, pancreatitis, choledocholithiasis, Mirizzi syndrome, genetic diseases, and malignancy.

### Ocular Diseases and Disorders

Any composition of the invention, including compositions described herein above or compositions that can for formulated from combining various embodiments of the present disclosure, can be used in treatment or prevention of ocular diseases and/or disorders including corneal opacification, glaucoma, age-related macular degeneration (AMD), cataract, and diabetic retinopathy (DR).

### Connective Tissue Diseases and Disorders

Any composition of the invention, including compositions described herein above or compositions that can for formulated from combining various embodiments of the present disclosure, can be used in treatment or prevention of connective tissue diseases and/or disorders including genetic disorders, autoimmune disorders, and cancer. Non-limiting examples of genetic connective tissue diseases and/or disorders include Marfan's syndrome, Loeys-Dietz syndrome, vascular Ehlers-Danlos syndrome (EDS), and osteogenesis imperfecta. Non-limiting examples of autoimmune connective tissue diseases and/or disorders include lupus scleroderma. Non-limiting examples of cancers associated with connective tissue diseases and/or disorders include soft tissue sarcoma.

In some embodiments, the connective tissue diseases and/or disorders are associated with other diseases and/or disorders as disclosed herein. For example, in some embodiments, the connective tissue diseases and/or disorders are associated with fibrosis, pulmonary fibrosis, interstitial lung disease, cancer, rheumatoid arthritis, scleroderma, polymyositis, and dermatomyositis.

### Cancer

In another embodiment, the compositions can be used in treatment or prevention of one or more cancers. Non-limiting examples of cancer include renal cell carcinoma, hepatocellular carcinoma, cholangiocarcinoma, breast cancer, and cutaneous squamous cell carcinoma. In one embodiment the cancer is associated with an organ or tissue including CNS, a lung, a liver, a heart, a kidney, a bowel, a stomach, one or more eyes, mediastinum, bone marrow, retroperitoneum, skin, an intestine, a joint, a reproductive organ, a prostate, a breast or a combination thereof.

In some embodiments, the compositions can be used in promotion of normal cell proliferation. In another embodiment, the compositions can be used in prevention of cell death in an organ. In some embodiments, the compositions can be used in reduction of cancerous tumor proliferation. In yet another embodiment, the compositions can be used to delay tumor growth. In some embodiments, the compositions can be used in inhibition of capase-3 activity. In another embodiment, the compositions can be used in sensitization of cancer cells to radiation therapy.

### Peroxisome Proliferator-Activated Receptors (PPARs)

Any composition of the invention, including compositions described herein above or compositions that can for formulated from combining various embodiments of the present disclosure, can be used in treatment or prevention of diseases and/or disorders mediated by PPARs including metabolic disease, NAFLD, metabolic syndrome, cardiovascular disease, insulin sensitivity, psoriasis, cancer, fibrosis, melanoma, neurodegenerative disorders, Huntington's disease, Parkinson's disease, Alzheimer's disease, inflammatory diseases, adipocyte differentiation, fertility or reproduction diseases, pain, inflammatory bowel disease, and obesity.

### Immunosuppressive Disease and Disorder

Any composition of the invention, including compositions described herein above or compositions that can for formulated from combining various embodiments of the present disclosure, can be used to induce immunosuppression. In various embodiments, immunosuppression is induced in an organ transplantation subject, such as a subject who has not yet received an organ transplant, who is receiving an organ transplant, or who has received an organ transplant. In some embodiments, the organ transplant is a renal transplant.

In some embodiments, the composition can be used to induce immunosuppression in a subject having an inflammatory and/or autoimmune disease. Non-limiting examples of inflammatory and/or autoimmune diseases include atopic dermatitis, hematologic malignancies, solid organ tumors such as breast carcinoma, atherosclerosis, multiple sclerosis, rheumatoid arthritis, type 1 diabetes mellitus, and inflammatory bowel diseases. Non-limiting examples of inflammatory bowel diseases include Crohn's disease, systemic lupus erythematosus, lupus nephritis, psoriasis, acne, asthma, and hidradenitis suppurative. Non-limiting examples of hematologic malignancies include Hodgkin's lymphoma, Non-Hodgkin's lymphoma, b-cell lymphomas, lymphocytic leukemia, multiple myeloma, and acute myeloid leukemia.

In some embodiments, the compositions can be used to reverse systemic immune activation in a subject in need thereof. In various embodiments, in response to administration of a composition, the subject experiences immunosuppression. In some embodiments, the immunosuppression is induced by the composition.

In various embodiments, compositions of the invention are administered in an amount sufficient to provide a daily 18-HEPE, 12-HEPE, and/or derivative thereof of about 50 mg to about 10000 mg, about 100 mg to about 7500 mg, or about 100 mg to about 5000 mg, for example, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, about 2500 mg about 2600 mg, about 2700 mg, about 2800 mg, about 2900 mg, about 3000 mg, about 3100 mg, about 3200 mg, about 3300 mg, about 3400 mg, about 3500 mg, 3600 mg, about 3700 mg, about 3800 mg, about 3900 mg, about 4000 mg, about 4100 mg, about 4200 mg, about 4300 mg, about 4400 mg, about 4500 mg, 4600 mg, about 4700 mg, about 4800 mg, about 4900 mg, about 5000 mg, about 5100 mg, about 5200 mg, about 5300 mg, about 5400 mg, about 5500 mg per day.

In one embodiment, the composition can be used in the treatment or preventing of atopic dermatitis, for example, mild to moderate atopic dermatitis. In one embodiment, the compositions is administered to the subject in need of such treatment in an amount of about 500 mg to about 3 g per day, about 1 g to about 2.5 g per day, about 1 g per day, or about 2 g per day. In one embodiment, the 18-HEPE, 12-HEPE, and/or derivative thereof is administered to the subject daily for a period of at least about 2 weeks, at least about 4 weeks, or at least about 8 weeks. In a related embodiment, upon treatment in accordance with the present invention, for example, over a period of about 1 to about 12 weeks, about 1 to about 8 weeks, or about 1 to about 4 weeks, the subject or subject group exhibits one or more of the following outcomes:

(a) a reduction in eczema area and severity index (EASI) score relative to baseline or placebo control;

(b) a reduction in percentage of area of an anatomical site affected by atopic dermatitis relative to baseline or control;

(c) a reduction in investigator's global assessment score relative to baseline or placebo control;

(d) a reduction in intensity of erythema, edema/population, oozing/crusts, excoriation, lichenification and/or dryness relative to baseline or placebo control;

(e) a reduction in erythema, edema/population, oozing/crusts, excoriation, lichenification and/or dryness relative to baseline or placebo control;

(f) a reduction in body surface area (BSA) affected by atopic dermatitis relative to baseline or placebo control;

(g) a reduction in loss of sleep relative to baseline or placebo control;

(h) a reduction in occurrence of pruritis (itch) relative to baseline or placebo control;

(i) a reduction in severity of pruritis as an average of the prior three days and/or nights on a visual analog scale;

(j) a reduction in SCORAD score relative to baseline or placebo control;

(k) an improved patient-oriented Eczema Measure (POEM) compared to baseline or placebo control;

(l) a reduction in number of days in the preceding week in which the subject reported that the skin was itchy due to eczema;

(m) a reduction in number of days in the preceding week in which the subject reported that their sleep was disturbed due to their eczema;

(n) a reduction in number of days in the preceding week in which the subject experienced skin bleeding;

(o) a reduction in number of days in the preceding week in which the subject experienced skin weeping or oozing clear fluid;

(p) a reduction in number of days in the preceding week in which the subject's skin cracked;

(q) a reduction in number of days in the preceding week in which the subject's skin flaked;

(r) a reduction in number of days in the preceding week in which the subject experienced dry skin;

(s) an increase in trans epidermal water loss compared to baseline or placebo control;

(t) an increase in plasma total and free 18-HEPE and/or 12-HEPE compared to baseline;

(u) a reduction in arterial blood pressure compared to baseline or placebo control.

In one embodiment, methods of the present invention comprise measuring baseline levels of one or more markers or parameters set forth in (a)-(u) above prior to dosing the subject or subject group. In another embodiment, the methods comprise administering a composition as disclosed herein to the subject after baseline levels of one or more markers or parameters set forth in (a)-(u) are determined, and subsequently taking an additional measurement of said one or more markers.

In another embodiment, upon treatment with a composition of the present invention, for example, over a period of about 1 to about 12 weeks, about 1 to about 8 weeks, or about 1 to about 4 weeks, the subject or subject group exhibits any 2 or more of, any 3 or more of, any 4 or more of, any 5 or more of, any 6 or more of, any 7 or more of, any 8 or more of, any 9 or more of, any 10 or more of, any 11 or more of, any 12 or more of, any 13 or more of, any 14 or more of, any 15 or more of, any 16 or more of, any 17 or more of, any 18 or more of, any 19 or more of, any 20 or more of, any 21 or more of, or all 22 of outcomes (a)-(u) described immediately above.

In another embodiment, upon treatment with a composition of the present invention, the subject or subject group exhibits one or more of the following outcomes:

(a) a reduction in eczema area and severity index (EASI) score relative to baseline or placebo control of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(b) a reduction in percentage of area of an anatomical site affected by atopic dermatitis relative to baseline or control of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(c) a reduction in investigator's global assessment score relative to baseline or placebo control of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(d) a reduction in intensity of erythema, edema/population, oozing/crusts, excoriation, lichenification and/or dryness relative to baseline or placebo control of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(e) a reduction in erythema, edema/population, oozing/crusts, excoriation, lichenification and/or dryness relative to baseline or placebo control of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(f) a reduction in body surface area (BSA) affected by atopic dermatitis relative to baseline or placebo control of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(g) a reduction in loss of sleep relative to baseline or placebo control of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(h) a reduction in occurrence of pruritis (itch) relative to baseline or placebo control of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(i) a reduction in severity of pruritis as an average of the prior three days and/or nights on a visual analog scale of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(j) a reduction in SCORAD score relative to baseline or placebo control of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(k) an improved patient-oriented Eczema Measure (POEM) compared to baseline or placebo control of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(l) a reduction in number of days in the preceding week in which the subject reported that their skin was itchy due to eczema of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(m) a reduction in number of days in the preceding week in which the subject reported that their sleep was disturbed due to their eczema of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(n) a reduction in number of days in the preceding week in which the subject experienced skin bleeding of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(o) a reduction in number of days in the preceding week in which the subject experienced skin weeping or oozing clear fluid of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(p) a reduction in number of days in the preceding week in which the subject's skin cracked of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(q) a reduction in number of days in the preceding week in which the subject's skin flaked of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(r) a reduction in number of days in the preceding week in which the subject experienced dry skin of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(s) an increase in trans epidermal water loss compared to baseline or placebo control of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(t) an increase in plasma total and free 18-HEPE and/or 12-HEPE compared to baseline of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(u) a reduction in mean arterial blood pressure of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at leas about 90% or at least about 95%.

In another embodiment, upon treatment with a composition of the present invention after a single dose administration or multiple dose administration, for example over a period of about 1 to about 12 weeks, about 1 to about 8 weeks, or about 1 to about 4 weeks, the subject or subject group exhibits any 2 or more of, any 3 or more of, any 4 or more of, any 5 or more of, any 6 or more of, any 7 or more of, any 8 or more of, any 9 or more of, any 10 or more of, any 11 or more of, any 12 or more of, any 13 or more of, any 14 or more of, any 15 or more of, any 16 or more of, any 17 or more of, any 18 or more of, any 19 or more of, any 20 or more of, any 21 or more of or all 22 of outcomes (a)-(u) described immediately above.

We claim:

1. A method of treating a disease and/or disorder in a subject in need thereof, the method comprising administering to the subject a composition comprising 18-HEPE, 12-HEPE, and/or derivative thereof.

2. The method of claim 1, wherein the disease and/or disorder is selected from the group consisting of skin, inflammatory, renal, rheumatic, respiratory, lung, cardiovascular, neurologic, fibrotic, liver, ocular, connective tissue, cancer, peroxisome proliferator activation receptor, urinary, and immunosuppressive disease and/or disorder.

3. The method of claim 2, wherein the skin disease and/or disorder is selected from the group consisting of acne vulgaris, acne rosacea, atopic dermatitis, psoriasis, pruritus/itch, radiation protection, dry skin, smooth skin, healthy skin, anti-aging, and photoprotection.

4. The method of claim 2, wherein the inflammatory disease and/or disorder is selected from the group consisting of chronic obstructive pulmonary disease (COPD), asthma, atopic dermatitis, and chronic rhinosinusitis.

5. The method of claim 2, wherein the renal disease and/or disorder is selected from the group consisting of kidney failure, acute kidney injury, chronic kidney disease, end stage renal disease, interstitial nephritis, kidney fibrosis, tubulointerstitial fibrosis, severe interstitial fibrosis, renal dysfunction, renal interstitial fibrosis, and polycystic kidney disease.

6. The method of claim 2, wherein the rheumatic disease and/or disorder is selected from the group consisting of ankylosing spondylitis, fibromyalgia, gout, infectious arthritis, lupus, osteoarthritis, polymyalgia rheumatica, psoriatic arthritis, reactive arthritis, rheumatoid arthritis, and scleroderma.

7. The method of claim 2, wherein the respiratory and/or lung disease and/or disorder is selected from the group consisting of inflammatory lung disease, respiratory tract infections, pleural cavity disease, pulmonary vascular disease, pneumonia, pulmonary embolism, lung cancer, idiopathic pulmonary fibrosis, sarcoidosis, mixed connective tissue disease, polymyositis, dermatomyositis, and systemic lupus erythematosus.

8. The method of claim 2, wherein cardiovascular disease and/or disorder is selected from the group consisting of acute cardiac ischemic events, acute myocardial infarction, angina, arrhythmia, atrial fibrillation, atherosclerosis, arterial fibrillation, cardiac insufficiency, cardiovascular disease, chronic heart failure, chronic stable angina, congestive heart failure, coronary artery disease, coronary heart disease, deep vein thrombosis, diabetes, diabetes mellitus, diabetic neuropathy, diastolic dysfunction in subjects with diabetes mellitus, edema, essential hypertension, eventual pulmonary embolism, fatty liver disease, heart disease, heart failure, homozygous familial hypercholesterolem ia (HoFH), homozygous familial sitosterolemia, hypercholesterolemia, hyperlipidemia, hypertension, hypertriglyceridemia, metabolic syndrome, mixed dyslipidemia, moderate to mild heart failure, myocardial infarction, obesity management, paroxysmal atrial/arterial fibrillation/flutter, paroxysmal supraventricular tachycardias (PSVT), particularly severe or rapid onset edema, platelet aggregation, primary hypercholesterolemia, primary hyperlipidemia, pulmonary arterial hypertension, pulmonary hypertension, recurrent hemodynamically unstable ventricular tachycardia (VT), recurrent ventricular arrhythmias, recurrent ventricular fibrillation (VF), ruptured aneurysm, sitosterolemia, stroke, supraventricular tachycardia, symptomatic atrial fibrillation/flutter, tachycardia, type-II diabetes, vascular disease, venous thromboembolism, ventricular arrhythmias, and other cardiovascular events.

9. The method of claim 2, wherein the neurologic disease and/or disorder is selected from the group consisting of catalepsy, epilepsy, encephalitis, meningitis, migraine, Huntington's, Alzheimer's, Parkinson's, and multiple sclerosis.

10. The method of claim 2, wherein the fibrotic disease and/or disorder is selected from the group consisting of systemic fibrosis (i.e., radiation fibrosis), liver fibrosis and/or cirrhosis, renal fibrosis, lung fibrosis and/or interstitial lung disease, skin fibrosis, cardiac fibrosis, ocular fibrosis, ocular disease, connective tissue disorders, myelofibrosis, cancers, idiopathic pulmonary fibrosis, and peyronie's disease.

11. The method of claim 2, wherein the liver disease and/or disorder is selected from the group consisting of fatty liver disease, non-alcoholic fatty liver disease (NAFLD), non-alcoholic steatohepatitis (NASH), alcoholic hepatitis, primary sclerosing cholangitis, primary biliary cholangitis, viral hepatitis (Chronic Hepatitis C, HBV), autoimmune hepatitis, Iatrogenic and/or drug induced liver injury, and hepatic veno-occlusive disease

12. The method of claim 2, wherein the ocular disease and/or disorder is selected from the group consisting of corneal opacification, glaucoma, age-related macular degeneration (AMD), cataract, and diabetic retinopathy (DR).

13. The method of claim 2, wherein the cancer is selected from the group consisting of renal cell carcinoma, hepatocellular carcinoma, cholangiocarcinoma, breast cancer, and cutaneous squamous cell carcinoma.

14. The method of claim 2, wherein the connective tissue disease and/or disorder is selected from the group consisting of genetic disorders, autoimmune disorders, and cancer.

15. The method of claim 2, wherein the peroxisome proliferator activation receptor disease and/or disorder is selected from the group consisting of metabolic disease, NAFLD, metabolic syndrome, cardiovascular disease, insulin sensitivity, psoriasis, cancer, fibrosis, melanoma, neurodegenerative disorders, Huntington's disease, Parkinson's disease, Alzheimer's disease, inflammatory diseases, adipocyte differentiation, fertility or reproduction diseases, pain, inflammatory bowel disease, and obesity

16. The method of claim 2, wherein the immunosuppressive disease and/or disorder atopic dermatitis, hematologic malignancies, solid organ tumors such as breast carcinoma, atherosclerosis, multiple sclerosis, rheumatoid arthritis, type 1 diabetes mellitus, and inflammatory bowel diseases.

17. The method of claim 2, wherein the urinary disease and/or disorder bladder cancer, cystocele, hematuria, interstitial cystitis, neurogenic bladder, Peyronie's disease, prostate disease, incontinence, urinary tract infection, and vasicoureteral reflux.

18. The method of claim 1, wherein the composition is administered to the subject in an amount sufficient to provide up to about 10 g of 18-HEPE, 12-HEPE, and/or a derivative thereof per day.

19. The method of claim 1, wherein the composition is administered in 1 to 8 capsules per day.

20. The method of claim 20, wherein each capsule comprises up to about 1 g of 18-HEPE, 12-HEPE, and/or a derivative thereof.

21. The method of claim 1, wherein the composition is administered to the subject for a period of at least about 1 week, at least about 2 weeks, at least about 4 weeks, at least about 6 weeks, at least about 8 weeks, or at least about 10 weeks.

22. The method of claim 1, wherein the composition is not encapsulated.

23. The method of claim 1, wherein the composition is administered by oral or topical administration.

24. A composition comprising 18-HEPE, 12-HEPE, and/or derivative thereof.

25. The composition of claim 24, wherein the derivative thereof is selected from the group consisting of an ester, a conjugate, a salt, or a combination thereof.

26. The composition of claim 24, wherein the composition is for oral or topical administration.

27. The composition of claim 24, wherein the composition is encapsulated in a capsule shell.

28. The composition of claim 24, wherein the composition is not encapsulated in a capsule shell.

29. The composition of claim 24, wherein the composition is in an oral dosage form.
